(19) United States
(12) Patent Application Publication
(10) Pub. No.: US 2025/0161594 A1
(43) Pub. Date: May 22, 2025

(54) NEEDLE SYSTEM

(71) Applicant: Eric Chou, Lake Forest, CA (US)

(72) Inventor: Eric Chou, Lake Forest, CA (US)

(21) Appl. No.: 18/511,922

(22) Filed: Nov. 16, 2023

(51) Int. Cl. A61M 5/46; A61M 5/32

(52) U.S. Cl. CPC A61M 5/46; A61M 5/3286; A61M 2207/00

(57) ABSTRACT

A needle system and method of manufacturing can include: providing a needle with a sharp tip; and coupling a body housing to the needle, the body housing comprising: angled inner walls configured to restrict lateral movement of the needle, and an extended depth limiter coupled to the needle, the extended depth limiter comprises a contact edge, the contact edge configured to be in contact with skin during operation to limit a depth of the needle within the skin.

### TECHNICAL FIELD

This disclosure relates to tattoo needles, more particularly to depth controlled needle systems.

### BACKGROUND

Body art, consisting of permanent makeup and tattoos, involves the insertion of pigment into the skin using a device with fine needles. Tattoo technology has progressed and developed over the millennia.

Prior developments in tattoo technology are exemplified by specialized tools having vibrating needles for puncturing and pigmenting skin. These prior developments provide adjustments including: an adjustable needle depth, which is how far the needle travels out of a tip; and a penetration depth, which is how far the tip of the needle penetrates the skin layer.

The needles move in an up and down motion creating punctures and simultaneous pigment placement within the skin. To achieve optimal tattoo results and ink retention, it is crucial to place the pigment at a specific depth beneath the surface layer of skin.

For instance, in the case of permanent makeup, the ideal depth would be between the epidermis and dermis. Prior developments relied on specialized needle cartridge designs to provide optimal depth of pigment placement.

These specialized needle cartridge designs consists of several components, including: a needle bar, a cartridge cap, a resilient barrier, a needle, a casing, and a needle tip. Even so, while the depth of the needles can be adjusted, the adjustment relies on the skill and experience of the user to manually guide and control the device.

Relying on skill and experience makes prior designs prone to operator error and misuse of the tools even when sophisticated mechanisms are used to control needle depth. Because of the high risks involved, many years are often required to become proficient in the tattooing trades.

One risk arising from the manual needle depth selection and hand movement control is pigment deposition on the wrong layer of the skin. Another risk arising from the manual needle depth selection and hand movement control occurs when a user attempts to stabilize the tattoo needle by using the tip as a support by resting it on the skin.

When the tip is used as a support, airflow required for proper insertion of the pigment can get clogged resulting in improper functioning of the tool and improper insertion of pigment. These previous developments in tattoo technology have failed to provide a consistent controllable depth to needle insertion without requiring difficult and expensive user training or improper functioning of tools.

Solutions have been long sought but prior developments have not taught or suggested any complete solutions, and solutions to these problems have long eluded those skilled in the art. Thus, there remains a considerable need for devices and methods that can provide a consistent and intuitively controllable needle depth.

### SUMMARY

A needle system and methods, providing a consistent and intuitively controllable needle depth, are disclosed. The needle system and methods manufacturing can include: providing a needle with a sharp tip; and coupling a body housing to the needle, the body housing comprising: angled inner walls configured to restrict lateral movement of the needle, and an extended depth limiter coupled to the needle, the extended depth limiter comprises a contact edge, the contact edge configured to be in contact with skin during operation to limit a depth of the needle within the skin.

Other contemplated embodiments can include objects, features, aspects, and advantages in addition to or in place of those mentioned above. These objects, features, aspects, and advantages of the embodiments will become more apparent from the following detailed description, along with the accompanying drawings.

### BRIEF DESCRIPTION OF THE DRAWINGS

The needle system is illustrated in the figures of the accompanying drawings which are meant to be exemplary and not limiting, in which like reference numerals are intended to refer to like components, and in which:

FIG. 1 is a cross-sectional view of a needle system in a first embodiment.

FIG. 2 is an isometric view of the needle system of FIG. 1.

FIG. 3 is a cross-sectional view of the needle system of FIG. 1 in a tattooing phase of operation.

FIG. 4 is a cross-sectional side view of a needle system in a second embodiment.

FIG. 5 is a cross-sectional front view of the needle system of FIG. 4.

FIG. 6 is a cross-sectional view of the needle system of FIG. 4 in a tattooing phase of operation.

FIG. 7 is a cross-sectional side view of the needle system of FIG. 4 in an inspection phase of operation.

FIG. 8 is an isometric view of the needle system of FIG. 4.

FIG. 9 is a control flow of a method of manufacturing the needle system.

### DETAILED DESCRIPTION

In the following description, reference is made to the accompanying drawings that form a part hereof, and in which are shown by way of illustration, embodiments in which the needle system may be practiced. It is to be understood that other embodiments may be utilized and structural changes may be made without departing from the scope of the needle system.

When features, aspects, or embodiments of the needle system are described in terms of steps of a process, an operation, a control flow, or a flow chart, it is to be understood that the steps can be combined, performed in a different order, deleted, or include additional steps without departing from the needle system as described herein.

The needle system is described in sufficient detail to enable those skilled in the art to make and use the needle system and provide numerous specific details to give a thorough understanding of the needle system; however, it will be apparent that the needle system may be practiced without these specific details.

In order to avoid obscuring the needle system, some well-known system configurations and descriptions are not disclosed in detail. Likewise, the drawings showing embodiments of the system are semi-diagrammatic and not to scale and, particularly, some of the dimensions are for the clarity of presentation and are shown greatly exaggerated in the drawing FIGs.

As used herein, the term “couple”, as in “coupling” or “coupled” is defined as direct or indirect physical contact between elements.

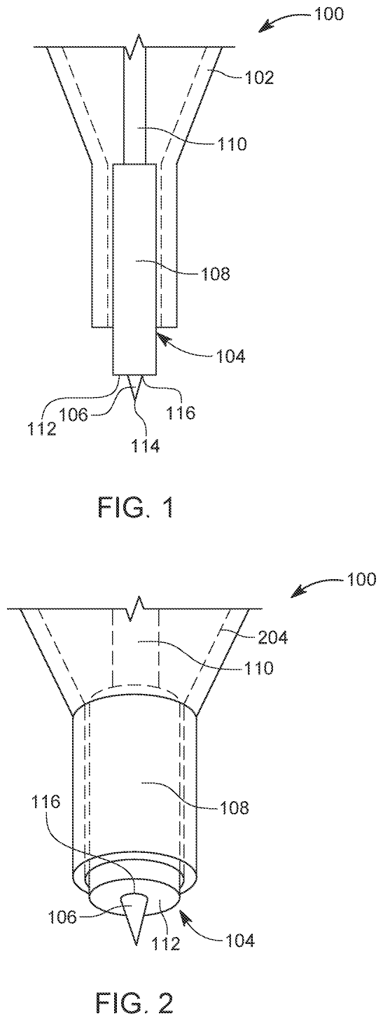

Referring now to FIG. 1, therein is shown a cross-sectional view of a needle system 100 in a first embodiment. The needle system 100 is shown having a body housing 102 around a needle 104.

The needle 104 is depicted with a sharp tip 106, a cylindrical bar 108 and a shank 110. The cylindrical bar 108 can include a contact edge 112 near the sharp tip 106. The cylindrical bar 108 can be understood as an extended depth limiter having the contact edge 112 to limit the depth of the sharp tip 106.

The contact edge 112 can be a portion of the cylindrical bar 108 facing skin while in use. The cylindrical bar 108 with the contact edge 112 can be understood as a depth limiter.

The depth limiter will prevent the sharp tip 106 from moving deeper into skin once the contact edge 112 hits the skin, thus limiting the depth of the needle 104. The sharp tip 106 extends a predefined distance beyond the contact edge 112.

The predefined distance, that the sharp tip 106 extends beyond the contact edge 112, can be the ink insertion depth within the skin and the maximum depth that the needle 104 can travel within the skin.

When the sharp tip 106 pierces through skin, the contact edge 112 will touch the skin and prevent further penetration into deeper skin layers, which creates a controlled and consistent penetration depth. This improved design will place the pigment at a fixed depth on every puncture, even with inconsistent hand motion.

This improved design ensures a consistent and controlled penetration depth, regardless of the amount of pressure applied by the user. The sharp tip 106 can be made of metal or other strong and sterilizable material.

The contact edge 112 of the cylindrical bar 108 only contacts the skin intermittently when the needle 104 is not striking, otherwise the cylindrical bar 108 is not in contact with the skin.

The body housing 102 can be in direct contact with the cylindrical bar 108 and can terminate near the contact edge 112. The cylindrical bar 108 can extend from and retract into the body housing 102; currently the cylindrical bar 108 is depicted in the extended phase of operation.

Near the shank 110, the body housing 102 can extend at an angle away from the cylindrical bar 108 and the shank 110. It is contemplated that the length of the cylindrical bar 108 can be longer or shorter than illustrated. The sharp tip 106 can include a corner 114 and a base 116.

The base 116 can be in direct contact with the contact edge 112 of the cylindrical bar 108. The corner 115 can be extended away from the contact edge 112 and the base 116.

The base 116 can have a similar diameter to that of the shank 110. The base 116 can have a diameter smaller than a diameter of the cylindrical bar 108 with the contact edge 112 extending horizontally out away from the base 116.

Referring now to FIG. 2, therein is shown an isometric view of the needle system 100 of FIG. 1. The cylindrical bar 108 is shown extended beyond the body housing 102. Angled inner walls 204 can contact the cylindrical bar 108 of the needle 104 near the shank 110.

The angled inner walls 204 can restrict lateral motion and provide lateral rigidity during operation allowing the needle 104 to only move in a controlled vertical direction while reducing contact area and friction. The contact edge 112 of the cylindrical bar 108 is shown in direct contact with the base 116 of the sharp tip 106 and can prevent the sharp tip 106 from penetrating into skin more than the height of the sharp tip 106.

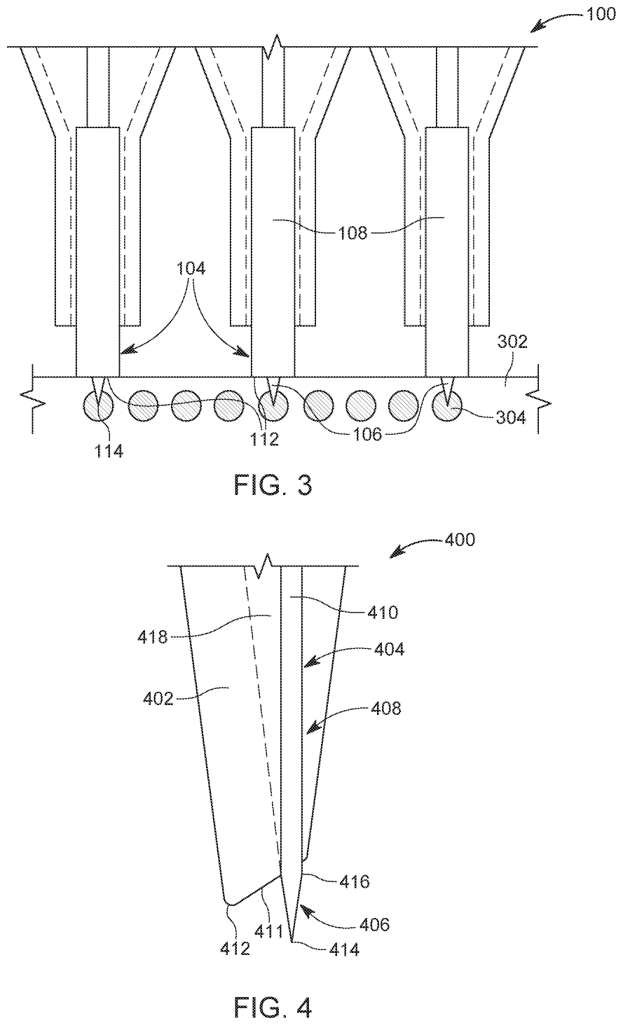

Referring now to FIG. 3, therein is shown a cross-sectional view of the needle system 100 of FIG. 1 in a tattooing phase of operation. The needle system 100 is shown having multiple needles 104 each having the cylindrical bar 108 with the contact edge 112.

The contact edge 112 of the cylindrical bar 108 is in direct contact with skin 302 of a patient. The sharp tip 106 of the needles 104 is shown penetrating the skin 302 from the corner 114 up to the contact edge 112 and depositing ink 304 in a uniform manner.

Referring now to FIG. 4, therein is shown a cross-sectional side view of a needle system 400 in a second embodiment. The needle system 400 is shown having a body housing 402 around a needle 404.

The needle 404 is depicted with a sharp tip 406 and a shank 410. The body housing 402 can include an angled back wall 411 extending away from the needle 404 and extending toward a contact edge 412 near the sharp tip 406.

The contact edge 412 can be a portion of the body housing 402 facing skin while in use. The body housing 402 with the angled back wall 411 can be understood as an extended depth limiter having the contact edge 412 to limit the depth of the sharp tip 406.

The depth limiter will prevent the sharp tip 406 from moving deeper into skin once the contact edge 412 hits the skin, and thus limits the depth of the needle 404. The sharp tip 406 can be extended and retracted relative to the contact edge 412.

The predefined distance, that the sharp tip 406 extends beyond the contact edge 412, can be the ink insertion depth within the skin and the maximum depth that the needle 404 can travel within the skin.

When the sharp tip 406 pierces through skin, the contact edge 412 will touch the skin and prevent further penetration into deeper skin layers, which creates a controlled and consistent penetration depth. This improved design will place the pigment at a fixed depth on every puncture, even with inconsistent hand motion.

This improved design ensures a consistent and controlled penetration depth, regardless of the amount of pressure applied by the user. The sharp tip 406 can be made of metal or other strong and sterilizable material.

The contact edge 412 of the body housing 402 contacts the skin continuously when the needle 404 in use as the needle system 400 can rest on the skin when the needle 404 is not striking. The needle 404 can extend from and retract into the body housing 402.

The sharp tip 406 can include a corner 414 and a base 416. The base 416 can be in direct contact with the shank 410. The corner 414 can be extended away from the shank 410 and the base 416.

The base 416 can have a similar diameter to that of the shank 410. The body housing 402 can be in direct contact with the shank 410 of the needle 404 near the sharp tip 406. Near the upper termination of the angled back wall 411, the body housing 402 can include angled inner walls 418.

The angled inner walls 418 can be in direct contact with the shank 410 of the needle 404 near the sharp tip 406 only and leave the remaining portion of the shank 410 free-floating as the angled inner walls 418 are angled and extend away from the shank 410. The angled inner walls 418 prevents lateral movement of the needle 404 for stability.

The angled back wall 411 allows the user to rest the contact edge 412 of the angled back wall 411 on the skin while operating the device. The angled back wall 411 controls how far the needle 404 penetrates through the skin, while the interior surface of the angled back wall 411, which faces the needle 404, provides support for the needle 404.

The angled back wall 411 also improves visibility and reduces pigment pooling in the treatment area. The needle can be extended and retracted beyond the contact edge 412 of the angled back wall 411, and the penetration depth of extension beyond the contact edge 412 can be adjusted. That is, a user can adjust the corner 114 of the needle 404 to extend a preselected distance past the contact edge 412 in order to target individual layers of skin and to adjust for varying thicknesses of skin.

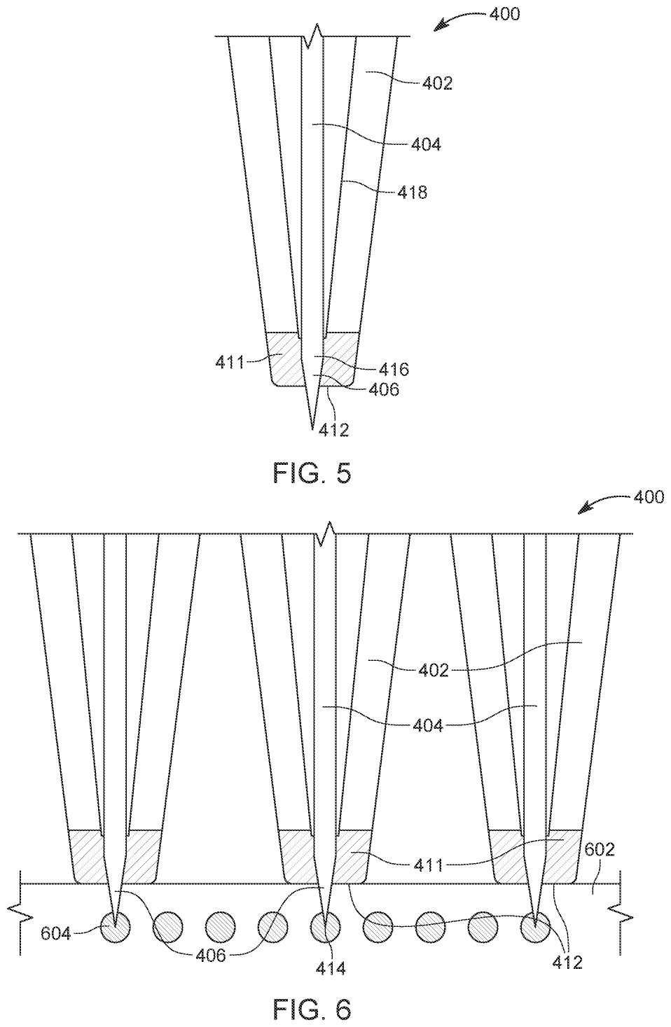

Referring now to FIG. 5, therein is shown a cross-sectional front view of the needle system 400 of FIG. 4. The body housing 402 is depicted having the angled back wall 411 terminating in the contact edge 412.

The sharp tip 406 of the needle 404 is shown extended down below the contact edge 412, but the needle 404 can be retracted during operation to be above the contact edge 412. The angled back wall 411 extends from the base 416 of the needle 404 to the contact edge 412.

The angled inner walls 418 are shown contacting the base 416 of the needle 404 and extending away from the needle 404 further from the sharp tip 406. The angled inner walls 418 can provide lateral support to keep the needle 404 from moving laterally during operation.

Referring now to FIG. 6, therein is shown a cross-sectional view of the needle system 400 of FIG. 4 in a tattooing phase of operation. The needle system 400 is shown having multiple needles 404 each extending from the body housing 102 having the angled back wall 411 with the contact edge 412.

The contact edge 412 is in direct contact with skin 602 of a patient. The sharp tip 406 of the needles 404 is shown penetrating the skin 602 from the corner 414 up to the contact edge 412 and depositing ink 604 in a uniform manner.

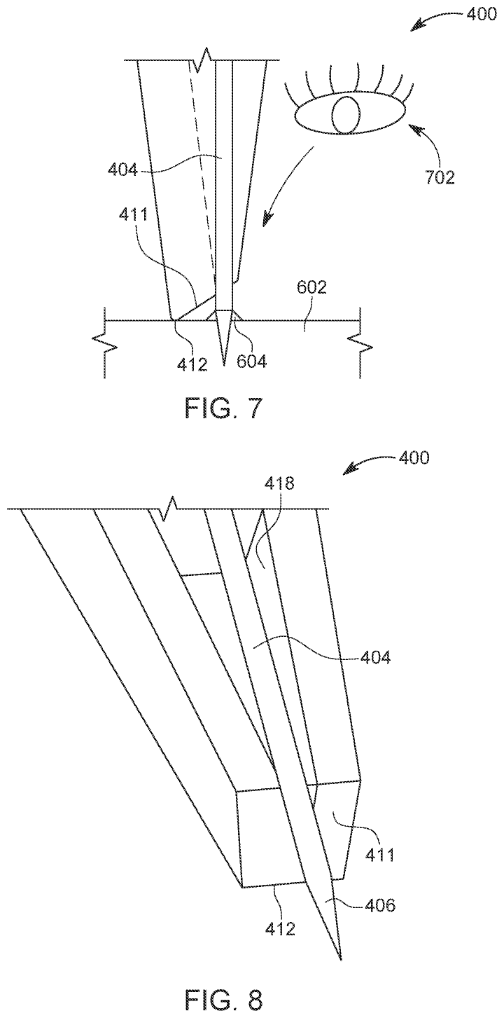

Referring now to FIG. 7, therein is shown a cross-sectional side view of the needle system 400 of FIG. 4 in an inspection phase of operation. One improvement that the needle system 400 provides to tattooing technologies is the viewing angle, which reveals the sharp tip of the needle 404 to the tattoo artist 702, and which provides the ability to more clearly see the tattooed skin while the needle 404 is penetrating the skin 602. Here, the angled back wall 411 allows the tattoo artist 702 to clearly see the needle 404, the ink 604, and the skin 602, real time, as the ink 604 is being deposited.

As will be appreciated, the angled inner walls 418 is angled not just side-to-side but also backward away from the needle 404. This backward slope to the angled inner walls 418 also stabilizes the needle 104 front-to-back.

Referring now to FIG. 8, therein is shown an isometric view of the needle system 400 of FIG. 4. The needle system 400 is shown with the needle 404 extended out away from the angled back wall 411. The angled inner walls 418 are angled and extended away from the needle 404 and the sharp tip 406 of the needle 404 extends below the contact edge 412.

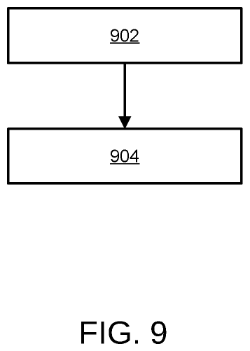

Referring now to FIG. 9, therein is shown a control flow of a method of manufacturing the needle system. The method of manufacturing can include: providing a needle with a sharp tip in a block 902; and coupling a body housing to the needle, the body housing comprising: angled inner walls configured to restrict lateral movement of the needle, and an extended depth limiter coupled to the needle, the extended depth limiter comprises a contact edge, the contact edge configured to be in contact with skin during operation to limit a depth of the needle within the skin in a block 904.

Thus, it has been discovered that the needle system furnishes important and heretofore unknown and unavailable solutions, capabilities, and functional aspects. The resulting configurations are straightforward, cost-effective, uncomplicated, highly versatile, accurate, sensitive, and effective, and can be implemented by adapting known components for ready, efficient, and economical manufacturing, application, and utilization.

While the needle system has been described in conjunction with a specific best mode, it is to be understood that many alternatives, modifications, and variations will be apparent to those skilled in the art in light of the preceding description. Accordingly, it is intended to embrace all such alternatives, modifications, and variations, which fall within the scope of the included claims. All matters set forth herein or shown in the accompanying drawings are to be interpreted in an illustrative and non-limiting sense.

What is claimed is:

1. A needle system comprising:
a needle with a sharp tip;
an extended depth limiter coupled to the needle, the extended depth limiter comprises a contact edge, the contact edge configured to be in contact with skin during operation to limit a depth of the needle within the skin.

2. The system of claim 1 wherein: the extended depth limiter is a cylindrical bar.

3. The system of claim 1 wherein: the extended depth limiter is an angled back wall.

4. The system of claim 1 wherein: the needle is adjustable with respect to the extended depth limiter.

5. The system of claim 1 wherein: the extended depth limiter is configured to only contact the skin intermittently during operation.

6. A needle system comprising:
a needle with a sharp tip; and
a body housing coupled to the needle, the body housing comprising:
angled inner walls configured to restrict lateral movement of the needle, and
an extended depth limiter coupled to the needle, the extended depth limiter comprises a contact edge, the contact edge configured to be in contact with skin during operation to limit a depth of the needle within the skin.

7. The system of claim 6 wherein: the extended depth limiter limits the depth of the needle independent of pressure applied to the body housing.

8. The system of claim 6 wherein: the body housing includes an angled back wall to prevent pooling of ink.

9. The system of claim 6 wherein: the contact edge is in direct contact with a base of the needle.

10. The system of claim 6 wherein: the extended depth limiter is an angled back wall providing a viewing angle revealing the sharp tip of the needle.

11. A method of manufacturing a needle system comprising:
providing a needle with a sharp tip;
coupling an extended depth limiter to the needle, the extended depth limiter comprises a contact edge, the contact edge configured to be in contact with skin during operation to limit a depth of the needle within the skin.

12. The method of claim 11 wherein: coupling the extended depth limiter includes coupling a cylindrical bar.

13. The method of claim 11 wherein: coupling the extended depth limiter includes coupling an angled back wall.

14. The method of claim 11 wherein: providing the needle includes providing the needle adjustable with respect to the extended depth limiter.

15. The method of claim 11 wherein: coupling the extended depth limiter includes coupling the extended depth limiter configured to only contact the skin intermittently during operation.

16. The method of claim 11 wherein: coupling the extended depth limiter includes coupling a body housing comprising the extended depth limiter to the needle, the body housing further comprising angled inner walls configured to restrict lateral movement of the needle.

17. The method of claim 16 wherein: coupling the extended depth limiter includes coupling the extended depth limiter configured to limit the depth of the needle independent of pressure applied to the body housing.

18. The method of claim 16 wherein: coupling the body housing comprising the extended depth limiter includes coupling an angled back wall to prevent pooling of ink.

19. The method of claim 16 wherein: coupling the extended depth limiter to the needle includes coupling the extended depth limiter having the contact edge in direct contact with a base of the needle.

20. The method of claim 16 wherein: coupling the extended depth limiter includes coupling the body housing comprising an angled back wall providing a viewing angle revealing the sharp tip of the needle.
